(19) United States
(12) Patent Application Publication
(10) Pub. No.: US 2026/0248492 A1
(43) Pub. Date: Aug. 27, 2026

(54) CLICK LOCK HANDLE FOR OVER THE SCOPE CLIP (OTSC)

(71) Applicant: BOSTON SCIENTIFIC SCIMED, INC., Maple Grove, MN (US)

(72) Inventors: Alexander COHEN, Austin, TX (US); Ryan Davis GILKEY, Austin, TX (US)

(21) Appl. No.: 19/465,202

(22) Filed: Jan. 30, 2026

Related U.S. Application Data
(60) Provisional application No. 63/762,969, filed on Feb. 25, 2025.

(51) Int. Cl. A61B 17/00; A61B 1/00; A61B 90/00

(52) U.S. Cl. CPC A61B 17/0057; A61B 1/00137; A61B 17/00234; A61B 90/08; A61B 2017/00367; A61B 2017/00584; A61B 2017/00623; A61B 2017/00646; A61B 2090/0811

(57) ABSTRACT

A clipping system includes a clip, an extension member, and a handle. The handle includes a body, a component and a flexible member. A proximal end of the member is coupled to the component so that longitudinal motion of the component along the body moves the member to transition the clip between insertion, initial deployment, and review configurations. The flexible member extends along a surface of the body to a distal end comprising a pin extending toward the surface which includes a track. The track has a non-uniform depth and variable depth features at locations along the track for resisting movement of the pin so that, as the component moves along the body and the pin travels within the track, the pin encountering the variable depth features along the track provides feedback regarding a position of the clip relative to the endoscope.

### PRIORITY CLAIM

The present disclosure claims priority to U.S. Provisional Patent Application Ser. No. 63/762,969 filed Feb. 25, 2025; the disclosure of which is incorporated herewith by reference.

### FIELD

The present disclosure relates to endoscopic devices and, in particular, relates to endoscopic clipping devices for treating tissue, for example, within the gastrointestinal tract.

### BACKGROUND

Physicians have become more willing to perform aggressive interventional and therapeutic endoscopic gastrointestinal (GI) procedures, which may increase the risk of perforating the wall of the GI tract or may require closure of the GI tract wall as part of the procedure. Such procedures may include, for example, the removal of large lesions, tunneling under the mucosal layer of the GI tract to treat issues below the mucosa, full thickness removal of tissue, treatment of issues on other organs by passing outside of the GI tract, and endoscopic treatment/repair of post-surgical issues (e.g., post-surgical leaks, breakdown of surgical staple lines, and anastomotic leaks). Currently, tissue may be treated via endoscopic closure devices including through-the scope clips or over-the-scope clips (OTSC).

Over-the-scope clips may be particularly useful for achieving closure of larger tissue defects. These clips are generally navigated to a target region in the human body in an insertion configuration in which the jaws of the clip are open, e.g., around an adapter fit around the distal end of a flexible endoscope. The clip may then be moved off the distal end of the adapter so that the natural bias of the clip causes the jaws to close, e.g., around target tissue. Some over-the-scope clips can be transitioned into a review configuration in which, after initially closing the clip over target tissue, the endoscope can be withdrawn proximally relative the clip (which remains in position closed over the target tissue) to widen the field of view of the endoscopic vision system and enable more extensive observation of the placement and/or position of the clip relative to the target tissue. If the placement of the clip is satisfactory, the operating physician can decouple the clip from the system by a release mechanism to fully deploy the clip.

The clip deployment operations described above, including transitioning the clip between its insertion configuration, initial deployment configuration, and review configuration and releasing the clip in the final deployment, are generally controlled by the operating physician via a handle. Existing handles for operating an OTSC currently lack features for precise control of these clip deployment operations, which are complicated by the long lengths of the control members required for these endoscopic tools.

### SUMMARY

The present disclosure relates to a clipping system for treating tissue. The system includes a clip mounted over a distal end of an endoscope in an insertion configuration in which jaws of the clip are in an open configuration; an extension member coupled at a distal end thereof to the clip, the extension member movable relative to the endoscope to move the clip off the distal end of the endoscope to transition the clip into an initial deployment configuration in which the jaws of the clip are in a closed configuration and to separate the clip in the closed configuration from the distal end of the endoscope by a distance to transition the clip into a review configuration; and a handle.

The handle includes comprising a handle body including an exterior surface shaped for gripping by an operating physician, the handle body including a longitudinal slot extending therethrough; a sliding component slidably coupled to the handle body by a body portion including a transverse portion extending through the longitudinal slot of the handle body, a proximal end of the extension member coupled to the sliding component so that longitudinal motion of the sliding component along the handle body moves the extension member to transition the clip between the insertion configuration, the initial deployment configuration and the review configuration; and a flexible member coupled at a proximal end to the sliding component and movable therewith, the flexible member extending along the exterior surface of the handle body to a distal end comprising a distal pin extending toward the exterior surface of the handle body.

The exterior surface of the handle body further includes a track sized and shaped for the distal pin of the flexible member to travel therewithin, the track comprising a depth that is non-uniform, the track including variable depth features at locations along the track for resisting movement of the distal pin within the track so that, as the sliding component moves along the handle body and the distal pin of the flexible member travels within the track, the distal pin encountering the variable depth features along the track provides feedback regarding a position of the clip relative to the endoscope.

In an embodiment, the flexible member is unflexed or at a minimum degree of flexing when the distal pin is engaged with the track in a location of maximum depth, wherein the distal pin moving to locations of lesser depth within the track causes the flexible member to flex radially outward relative to the handle body.

In an embodiment, the track includes a locking location wherein, when the distal pin is within the locking location, the distal pin is impeded from traveling outside the locking location such that, absent a force being applied to the sliding component by a user, the distal pin remains in the locking location and the clip remains in position relative to the endoscope.

In an embodiment, the track includes a proximal portion shaped in a loop, wherein the proximal end of the flexible member is rotatably coupled to the sliding component to permit the flexible member to rotate out of longitudinal alignment with the handle body and permit the distal pin to travel within the loop.

In an embodiment, the depth of the proximal portion of the track is configured to urge the distal pin to travel in only one direction around the loop.

In an embodiment, first parts of the loop are angled to reduce the depth of the track and second parts of the loop include drop-offs to increase the depth of the track such that, after the distal pin travels in a first direction around the loop past a drop-off, the distal pin is restricted from traveling in a second direction around the loop opposite the first direction.

In an embodiment, the locking location is within the proximal portion of the track and corresponds to the insertion configuration of the clip, the locking location being distal to parts of the loop on either side the locking location so that the distal pin can exit the locking location by the user withdrawing the sliding component further proximally, causing the distal pin to travel proximally to a drop-off, whereupon the distal pin can travel distally out of the loop.

In an embodiment, the track includes a ridged portion comprising a series of ridges so that, as the distal pin travels along the ridged portion, tactile feedback is imparted to the handle.

In an embodiment, the distal pin traveling distally over the ridged portion corresponds to transitioning the clip from the initial deployment configuration to the review configuration.

In an embodiment, the clipping system further includes a release member in a retaining position configured to retain a coupling of the clip with the clipping system, the release member movable to a release position in which the release member is withdrawn proximally to remove the coupling and the clip to decouple from the clipping system.

In an embodiment, the clipping system further a deployment feature to which a proximal end of the release member is fixed, the deployment feature including a switch operable to withdraw the release member proximally relative to the clip.

In an embodiment, the clipping system further includes a spring comprising a proximal end coupled to the deployment feature and a distal end coupled to the sliding component so that motion of the sliding component is partially decoupled from motion of the deployment feature.

In an embodiment, the sliding component includes a back portion extending proximally from the body portion and a proximal ring at a proximal end, the deployment feature positioned between the proximal ring and the body portion of the sliding component restricting the relative motion between the deployment feature and the sliding component.

In an embodiment, the back portion of the sliding component includes a slot, a pin extending through the slot and connecting to the deployment feature to constrain a relative distance between the deployment feature and the body portion of the sliding component.

In an embodiment, the relative distance between the deployment feature and the body portion of the sliding component can vary during operation of the clip.

In addition, a method for treating tissue includes inserting, to a target area in a body lumen, a clip mounted over a distal end of an endoscope in an insertion configuration in which first and second jaws of the clip are in an open configuration, an extension member coupled at a distal end thereof to the clip, the extension member movable relative to the endoscope by a handle comprising a handle body including an exterior surface shaped for gripping by an operating physician, the handle body including a longitudinal slot extending therethrough, the handle comprising a sliding component slidably coupled to the handle body by a body portion including a transverse portion extending through the longitudinal slot of the handle body, a proximal end of the extension member coupled to the sliding component so that longitudinal motion of the sliding component along the handle body moves the extension member, the handle comprising a flexible member coupled at a proximal end to the sliding component and movable therewith, the flexible member extending along the exterior surface of the handle body to a distal end comprising a distal pin extending toward the exterior surface of the handle body, wherein the exterior surface of the handle body further includes a track sized and shaped for the distal pin of the flexible member to travel therewithin, the track comprising a depth that is non-uniform, the track including variable depth features at locations along the track for resisting movement of the distal pin within the track so that, as the sliding component moves along the handle body and the distal pin of the flexible member travels within the track, the distal pin encountering the variable depth features along the track provides feedback regarding a position of the clip relative to the endoscope; drawing tissue between the first and second jaws of the clip; sliding the sliding component distally to move the extension members distally to move the clip distally off of the distal end of the endoscope to transition the clip into an initial deployment configuration in which the first and second jaws of the clip are in a closed configuration around the tissue; and sliding the sliding component further distally to move the extension members further distally to separate the clip in the closed configuration from the distal end of the endoscope by a distance to transition the clip into a review configuration. The distal pin encounters the variable depth features along the track during the sliding of the sliding component to provide feedback regarding transitions between the insertion configuration, the initial deployment configuration and the review configuration.

In an embodiment, the flexible member is unflexed or at a minimum degree of flexing when the distal pin is engaged with the track in a location of maximum depth, wherein the distal pin moving to locations of lesser depth within the track causes the flexible member to flex radially outward relative to the handle body.

In an embodiment, the track includes a locking location wherein, when the distal pin is within the locking location, the distal pin is impeded from traveling outside the locking location such that, absent a force being applied to the sliding component by a user, the distal pin remains in the locking location and the clip remains in position relative to the endoscope.

In an embodiment, the track includes a proximal portion shaped in a loop, wherein the proximal end of the flexible member is rotatably coupled to the sliding component to permit the flexible member to rotate out of longitudinal alignment with the handle body and permit the distal pin to travel within the loop.

In an embodiment, the track includes a ridged portion comprising a series of ridges so that, as the distal pin travels along the ridged portion, tactile feedback is imparted to the handle.

### BRIEF DESCRIPTION OF DRAWINGS

FIG. 1 shows a perspective view of a handle for controlling the deployment of an over-the-scope clip according to various exemplary embodiments.

FIG. 2 shows a perspective view of a handle body of the handle of FIG. 1.

FIG. 3 shows a front view of the handle body of FIG. 2.

FIG. 4 shows a side view of the handle body of FIG. 2.

FIG. 5 shows a perspective view of a sliding component of the handle of FIG. 1.

FIG. 6 shows a perspective view of the sliding component of FIG. 5 with a flexible member coupled thereto.

FIG. 7 shows a front view of the sliding component and the flexible member of FIG. 6.

FIG. 8 shows a perspective view of the flexible member of FIG. 6.

FIG. 9 shows a side view of a track formed in the handle body of FIG. 2.

FIG. 10 shows a perspective view of a ridged portion of the track of FIG. 9.

FIG. 11 shows a side view of a proximal portion of the track of FIG. 9 comprising a loop.

FIG. 12 shows a perspective view of the proximal portion of the track of FIG. 11.

FIG. 13 shows a side view of the flexible member of FIG. 8 engaged with a first part of the proximal portion of the track of FIG. 9.

FIG. 14 shows a side view of the flexible member of FIG. 8 engaged with a fourth part of the proximal portion of the track of FIG. 9 at the top of a proximal stroke.

FIG. 15 shows a side view of the flexible member of FIG. 8 engaged with a locking position of the proximal portion of the track of FIG. 9.

FIG. 16 shows a side view of the flexible member of FIG. 8 engaged with a seventh part of the proximal portion of the track of FIG. 9 in an unlocked position.

FIG. 17 shows a side view of the flexible member of FIG. 8 engaged with a ridged portion of the track of FIG. 9.

FIG. 18 shows a side view of the flexible member of FIG. 8 engaged with the ridged portion of the track of FIG. 9 at a bottom of a distal stroke.

FIG. 19 shows a perspective view of a deployment feature of the handle of FIG. 1 in an undeployed position.

FIG. 20 shows a perspective view of a deployment feature of the handle of FIG. 1 in a deployed position.

FIG. 21 shows a front view of the handle of FIG. 1 with a spring coupling the sliding component of FIG. 5 and the deployment feature of FIG. 19 in an uncompressed state.

FIG. 22 shows a front view of the handle of FIG. 1 with the spring coupling the sliding component of FIG. 5 and the deployment feature of FIG. 19 in a compressed state.

FIG. 23 shows a perspective view of a clipping system compatible with the handle of FIG. 1.

FIG. 24 shows a perspective view of a clip of the clipping system of FIG. 23.

### DETAILED DESCRIPTION

The present disclosure may be further understood with reference to the following description and the appended drawings, wherein like elements are referred to with the same reference numerals. The present disclosure relates to a handle of an endoscopic system which, in some embodiments, comprises an over-the-scope endoscopic clipping system. In some aspects of the present disclosure, handle features are described for enhancing user control of the clipping system. In an embodiment, the exemplary handle includes features for tracking the position of one or more control members extending from the handle to the clip to enable the user to precisely control the configuration of the clip. In another embodiment, the exemplary handle includes features for locking a position of the control member(s). In other aspects, handle features are described for compensating for relative motion between first control members and second control members that may naturally occur when navigating the clip via the endoscope through tortuous paths of body lumens.

It should be understood that some aspects of the present disclosure relate to over-the-scope endoscopic clipping systems in which an initial placement of a clip may be viewed and, if desired, the clip may be reopened and repositioned prior to a final deployment of the clip. These clipping systems include control features for transitioning the clip between an insertion configuration, wherein the clip is mounted to an adapter on the distal end of the endoscope with the clip jaws in an open configuration, an initial deployment configuration, wherein the clip moves off the adapter and the clip jaws close around target tissue distal and adjacent to the adapter, and a review configuration, wherein the clip in the closed configuration is separated from the adapter by a distance (e.g., the endoscope is withdrawn proximally relative to the clip) to widen the field of view of the endoscopic vision system and enable more extensive observation of the placement and/or position of the clip relative to the target tissue.

An operating physician can transition the clip between these configurations by moving one or more control members (referred to herein as “first control members” or “extension members” and, in one example, corresponding to extension members 230 described below in the exemplary clipping system 200) proximally and/or distally by a control interface on a handle of the system. These control member(s) are coupled at a proximal end to the control interface, e.g., a sliding component coupled to the handle body, and at a distal end to the clip such that distal motion of the sliding component translates to distal motion of the clip (e.g., from the insertion configuration to the initial deployment configuration or from the initial deployment configuration to the review configuration) and proximal motion of the sliding component translates to proximal motion of the clip (e.g., from the initial deployment configuration to the insertion configuration or from the review configuration to the initial deployment configuration).

It should be further understood that some aspects of the present disclosure relate to over-the-scope endoscopic clipping systems including control features for releasing the clip from the system in a final deployment. An operating physician can release the clip by moving one or more further control members (referred to herein as “second control members” or “release members” and, in one example, corresponding to release members 250 described below in the exemplary clipping system 200) by a further control interface on the handle of the system. These control member(s) are coupled at a proximal end to the control interface, e.g., a release mechanism, and at a distal end to the clip such that triggering the release mechanism functions to release the clip, e.g., by retracting the release members. It will be understood by those skilled in the art that the terms proximal and distal, as used herein, are intended to refer to a direction toward (proximal) and away from (distal) a user of the device.

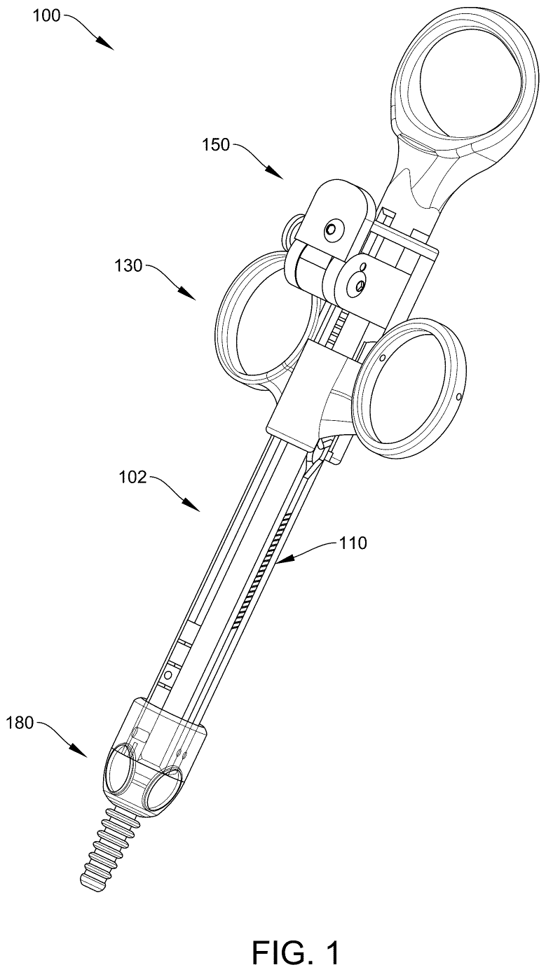

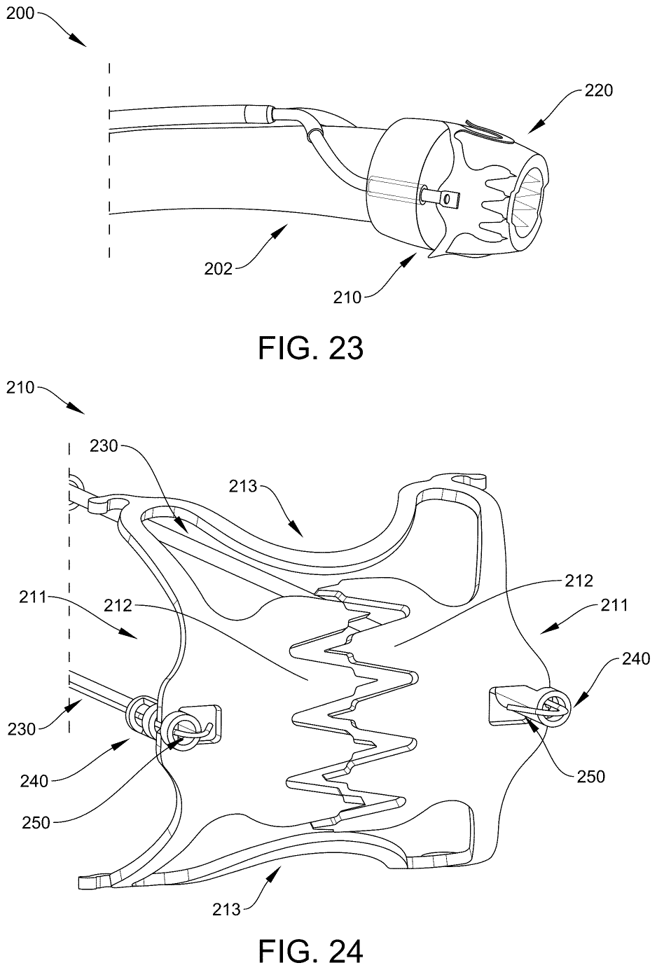

FIGS. 23-24 show a clipping system 200 according to one example. It should be understood that the exemplary clipping system 200 is described briefly to introduce one example of a clipping system and illustrate how such a clipping system can be implemented (i.e., operated with) with handle features as described in further detail below. In particular, the exemplary clipping system 200 can be implemented with the handle 100 described in detail below with regard to FIGS. 1-22. However, those skilled in the art will understand that the handle functionalities described herein are not limited to implementation with the clipping system 200. For example, the handle functionalities described herein can be implemented with various types of clip deployment functionalities. In some cases, the handle functionalities described herein can be implemented with different types of endoscopic treatment systems—i.e., with end effectors other than hemostatic clips.

As shown in FIG. 23, the clipping system 200 comprises a clip 210 coupled to an adapter 220 configured to be mounted over the distal end of an endoscope 202 or a similar insertion device (e.g., with a friction fit). The adapter 220 may be formed, for example, as a cap of transparent material so that the vision system and working channel(s) of the endoscope 202 are unimpaired and so that the working channel(s) and the vision system are aimed into an open lumen of the adapter 220. The clip 210 is configured to be moved proximally and distally over the adapter 220 via extension members 230, to which the clip 210 is releasably coupled. In the example of FIG. 23, the clip 210 is in the insertion configuration wherein the clip 210 stretched open around the adapter 220 against a natural bias urging the jaws of the clip 210 together. In the insertion configuration, the clip 210 is advanced into the body by moving the endoscope 202 on which the adapter 220 is mounted through one or more body lumens to a target location within the human body.

As shown in FIG. 24, the clip 210 includes two jaws 211 connected to one another via hinges 213. In the example of FIG. 24, the clip is in a closed configuration. The hinges 213 can be spring biased, biasing the jaws 211 toward the closed configuration in which gripping features 212 of the jaws 211 are moved toward one another such that target tissue can be gripped therebetween. In the present example, the gripping features 212 comprise teeth sized and shaped so that, in the closed configuration, the teeth of one of the jaws 211 intermesh with the teeth of the other jaw 211.

In this example, the clip 210 is coupled to the extension members 230 via distal couplings 240 and release members 250, described in greater detail below. In operation, the adapter 220 is mounted over the distal end of the endoscope 202 and the endoscope 202 is navigated to a target site with the clip 210 in the insertion configuration. In the insertion configuration, the clip 210 is mounted over the adapter 220 with the jaws 211 spread apart from one another so that tissue drawn into the lumen of the adapter 220 is positioned between the open jaws 211. A target portion of tissue can be drawn into the lumen of the adapter 220, e.g., by suction, grasper, etc. applied through a working channel (not shown) of the endoscope that opens into the lumen of the adapter 220. The user may then move the clip 210 from the insertion configuration toward the initial deployment configuration by moving the extension members 230 distally relative to the adapter 220. This pushes the clip 210 distally off of the adapter 220 permitting the natural bias of the clip 210 to move the jaws 211 to the closed configuration in which the jaws 211 are drawn together to grip the tissue that was drawn into the adapter 220.

After tissue has been preliminarily clipped by the jaws 211 in the initial deployment configuration, the endoscope 202 may be moved proximally relative to the clip 210 to the review configuration by withdrawing the endoscope 202 proximally relative to the extension members 230 so the clip 210 remains coupled to the rest of the system while clipped to the tissue so that the increased distance between the endoscope 202 and the clip 210 enhances the user's ability to observe the position of the clip 210 in regard to the target tissue. The review configuration, as shown in Fig. Y, widens the field of view of the endoscopic vision system while also allowing for movement of the endoscope 202 relative to the clip 210 to enable more extensive observation of the placement and/or position of the clip 210 relative to the target tissue from various viewing angles while maintaining the coupling of the clip 210 to the extension members 230.

If the user determines the position of the clip 210 is incorrect or sub-optimal, the user may transition the clip 210 back to the initial deployment configuration by moving the endoscope 202 distally relative to the clip 210 to position the distal end of the adapter 220 adjacent to the clip 210, i.e., by sliding the endoscope 202 distally over the extension members 230. Transitioning the clip 210 back into the insertion configuration comprises withdrawing the extension members 230 proximally while holding the endoscope 202 in position. To do this, the clip 210 is held in contact with the distal end of the adapter 220 while the user pulls the clip 210 proximally against the tapered distal end of the adapter 220 to force the clip 210 to open, releasing the previously clipped tissue as the clip 210 is drawn back onto the adapter 220 in the insertion configuration with the jaws 211 spread apart from one another. The user may then reposition the endoscope 202 and the clip 210 and repeat these steps to adjust the placement and/or position of the clip 210 relative to the target site until the clip 210 is placed to the satisfaction of the user. When the user is satisfied that the clip 210 is positioned as desired, the user separates the clip 210 from the rest of the clipping system 200 so that the clip 210 may be left in place clipped over desired tissue in a final deployment while the rest of the clipping system 200 is withdrawn from the body.

In this example, the mechanism for releasing the clip in the final deployment comprises withdrawing the release members 250 proximally relative to the extension members 230. The release mechanism of this example includes a distal coupling 240 fixed to the distal end of the extension members 230 to which the jaws 211 are releasably coupled via the release members 250. In particular, the distal coupling 240 is shaped with a recess in which a side portion of the jaws 211 is received. The release members 250 extend through a channel of the distal coupling 240 and around the side portion of the jaws 211 to retain the side portion of the jaws 211 within the recess. The release members 250 extend past a distal end of the distal coupling to an unanchored distal end.

After assembly of the device, and during initial deployment operations, the release members 250 remain in a substantially fixed position relative to the extension members 230 and the distal couplings 240 such that the clip 210 remains coupled to the extension members 230 and the remainder of the clipping system 200 until the physician determines to finally deploy the clip 210. When the physician determines the placement of the clip 210 is satisfactory, the release members 250 are retracted proximally relative to the extension members 230 such that the side portions of the jaws 211 are no longer restrained by the release members 250 within the recess. The extension members 230 are then retracted along with the distal couplings 240 to decouple the clip 210 from the distal couplings 240, leaving the clip 210 in place over the target tissue.

Accordingly, a clipping system can include at least two different types of control members for operating the clip. As described above, clipping systems with the capacity to space a preliminarily deployed clip from the endoscope by an increased distance to review the placement of the clip (e.g., the review configuration of the clipping system 200 described above) can include control members (e.g., extension members 230 of the clipping system 200 described above) controlled by an operating physician to move distally and/or proximally to transition the clip between an insertion configuration, an initial deployment configuration, and a review configuration. Additionally, these clipping systems can include control members (e.g., release members 250 of the clipping system 200 described above) controlled by an operating physician to trigger or otherwise facilitate the separation of the clip from the remainder of the system for final deployment.

The above-described control functionalities are generally implemented by a handle configured to be gripped and manipulated by the operating physician. Transitioning the clip between the insertion configuration, the initial deployment configuration, and the review configuration are controlled, in this embodiment, by a linear sliding feature of the handle such as, e.g., a sliding component slidably coupled to the handle body to which proximal ends of the extension members are fixed. In current clipping systems, the operating physician typically holds the sliding component for the duration of the procedure to prevent unwanted movement of the extension members that could cause an unintended transition of the clip between its various operating configurations.

According to various exemplary embodiments described herein, a handle is described including features for improving a user experience in deploying a clip. It should be understood that various features described herein can be utilized in other types of medical devices and are not limited to hemostasis clips.

In one aspect of these exemplary embodiments, the handle includes a flexible member comprising a proximal end coupled to a sliding component and a distal end slidably received within a track formed in the handle body. The flexible member may be referred to herein as a flexure. In an embodiment, the distal end of the flexure comprises a pin sized and shaped to travel along the track. In an embodiment, the track generally comprises a slot shaped with features having a variable depth, wherein certain locations within the slot are shaped to guide or restrict the motion of the pin therein. Due to the coupling of the flexure to the sliding component, the motion of the pin within the slot is directly correlated to the motion of the extension members and, by extension, the configuration of the clip at the distal end of the device. In an embodiment, the track is shaped with a locking location that retains the pin in the location, such that the clip is retained in a desired configuration (e.g., the insertion configuration) without requiring active user control to maintain the configuration, e.g., to prevent the sliding component from slipping relative to the handle body (thus, transitioning the clip toward a different operating configuration) if the user if not actively controlling the location of the sliding component relative to the handle body.

In another embodiment, the track includes ridged or grooved features that provide a small resistive force to the pin traveling within the slot, functioning as a feedback mechanism as the pin travels over the ridges. The pin is allowed to travel within the track due to the flexible nature of the flexure. By bending slightly as the distal pin encounters these features, the flexure permits the distal pin to move radially outward or inward to conform to the variable depth of the slot.

In another aspect of these exemplary embodiments, the handle includes a deployment feature for actuating the final deployment of the clip. In an embodiment, the deployment feature is coupled to the handle body and slidable within the slot of the handle body. In an embodiment, the deployment feature is proximal to the body of the sliding component and coupled thereto so that the sliding component and the deployment feature move in coordination along the handle body. In an embodiment, proximal ends of second control members are fixed to the deployment feature. In an embodiment, the second control members comprise release members coupled to the clip via distal couplings. However, those skilled in the art will understand that other deployment mechanisms for the clip can be used with the exemplary deployment feature for the handle.

In general, the deployment feature maintains the second control members in position relative to the clip (and the first control members) until the deployment feature is actuated to finally deploy the clip. In an embodiment, the second control members are fixed to a rotatable switch of the deployment feature so that rotating the switch draws the second control members proximally relative to the clip. However, it should be understood that different deployment mechanisms may be used in the present handle. In general, any deployment mechanism can be used in which secondary control members are to be held in position relative to primary control members until the user determines to finally deploy the clip, at which time the deployment mechanism draws the secondary control members proximally.

In another aspect of these exemplary embodiments, the handle includes a spring having a proximal end coupled to the deployment feature and a distal end coupled to the sliding component. This spring is referred to here as a compensation spring. In an embodiment, the spring functions to partially decouple the motion of the sliding component from the deployment feature to compensate for slight changes in the relative lengths of various components of the device as the device is bent in different directions and around different bending radii along the path to a position adjacent to the target tissue.

FIGS. 1-22 show a handle 100 for controlling the deployment of an over-the-scope clip according to various exemplary embodiments. As shown in FIG. 1, the handle 100 generally includes a handle body 102 including a track 110 on its exterior surface. The handle 100 further includes a sliding component 130 and a deployment feature 150 slidably coupled to the handle body 102. A distal end of the handle 100 includes an adapter 180 (shown transparently) enabling connections to additional components, e.g., a flexible elongate member comprising a channel carrying the control members of the handle 100, to be described in greater detail below. The adapter 180 may correspond to existing adapters and its configuration is generally outside the scope of the present disclosure.

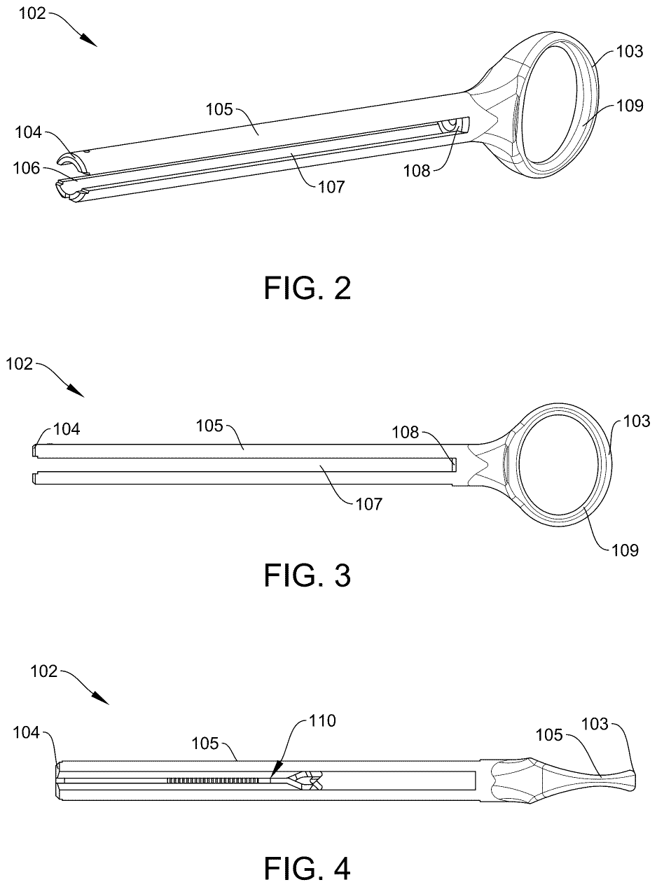

As shown in FIGS. 2-4, the handle body 102 extends from a proximal end 103 to a distal end 104. The proximal end 103 comprises a proximal ring 109 sized and shaped to receive a thumb of the user. The handle body 102 generally comprises a longitudinal portion 105 configured to be gripped by a user. In this example, the longitudinal portion 105 is generally cylindrical, however, this is not required. In an embodiment, a longitudinal channel 106 extends through the longitudinal portion 105. Certain components of the handle 100, including the cylindrical portion 136 on the transverse portion 135 of the sliding component 130, the cylindrical portion on the transverse portion of the deployment feature 150, and a spring 182 coupling the sliding component 130 and the deployment feature 150 may be sized and shaped to fit in the channel 106, to be described in greater detail below. In an embodiment, a slot 107 extends longitudinally through the longitudinal portion 105 from a proximal end 108 adjacent to the proximal ring 109 through the distal end 104 of the handle body 102.

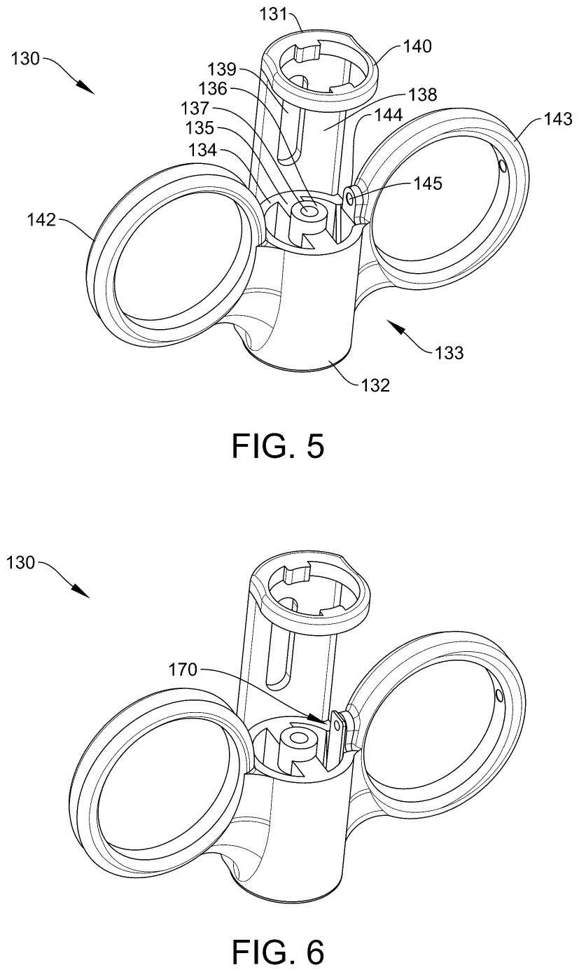

As shown in FIGS. 5-7, the handle 100 further includes a sliding component 130 slidingly coupled to the handle body 102. The sliding component 130 can travel distally and proximally along the handle body 102 to operate first control members (not shown) fixed to and extending distally from the sliding component 130. In particular, the first control members of this embodiment correspond to the extension members 230 of the clipping system 200 that transition the clip 210 between the insertion configuration, the initial deployment configuration and the review configuration. The sliding component 130 includes first and second rings 142, 143 for configured to be gripped by fingers of the operating physician such that spreading the thumb from the forefingers moves the sliding component 130 distally along the handle body 102 and withdrawing the forefingers toward the thumb moves the sliding component 130 proximally along the handle body 102.

The sliding component 130 comprises a body portion 133 shaped in this embodiment as a circumferential ring 134 external to the handle body 102 with a transverse portion 135 bisecting the ring 134 that is slidably received in the slot 107. The transverse portion 135 further includes a cylindrical portion 136 that is slidably received in the channel 106 of the handle body 102. Accordingly, the cross-sectional geometry of the sliding component 130 corresponds to the channels within the handle body 102. The cylindrical portion 136 includes a channel 137 through which second control members can pass from the deployment feature 150.

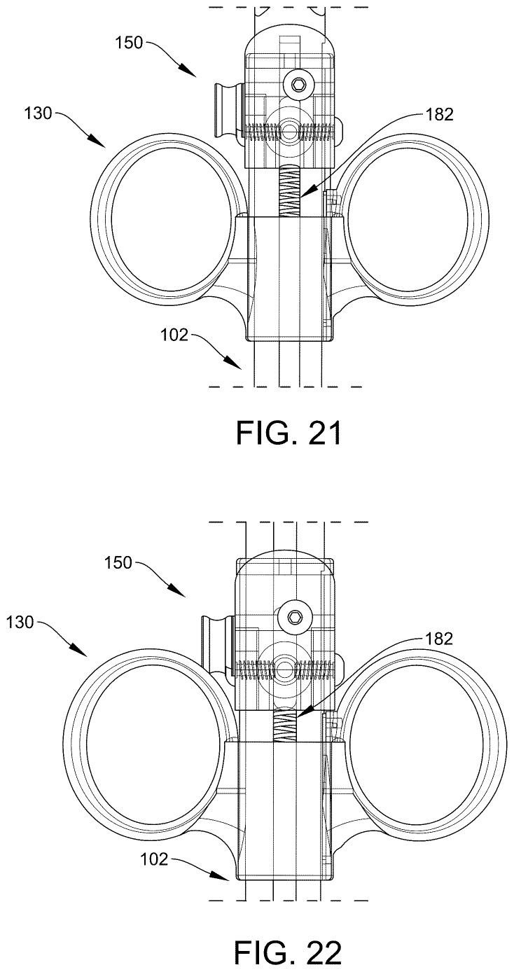

The first ring 142 and the second ring 143 are configured to be gripped by the fingers of the operating physician and extend radially outward from opposing sides of the body portion 133. A back portion 138 extending proximally from a proximal end of the body portion 133 is relatively thin. In this example, the back portion 138 includes a longitudinal slot 139 and a proximal end 131 of the sliding component 130 comprises a proximal ring 140 at the proximal end of the back portion 138. The deployment feature 150 is slidably coupled to the handle body 102 between the proximal ring 140 and the body portion 133 of the sliding component 130. These features will be explained in greater detail below with regard to FIGS. 21-22.

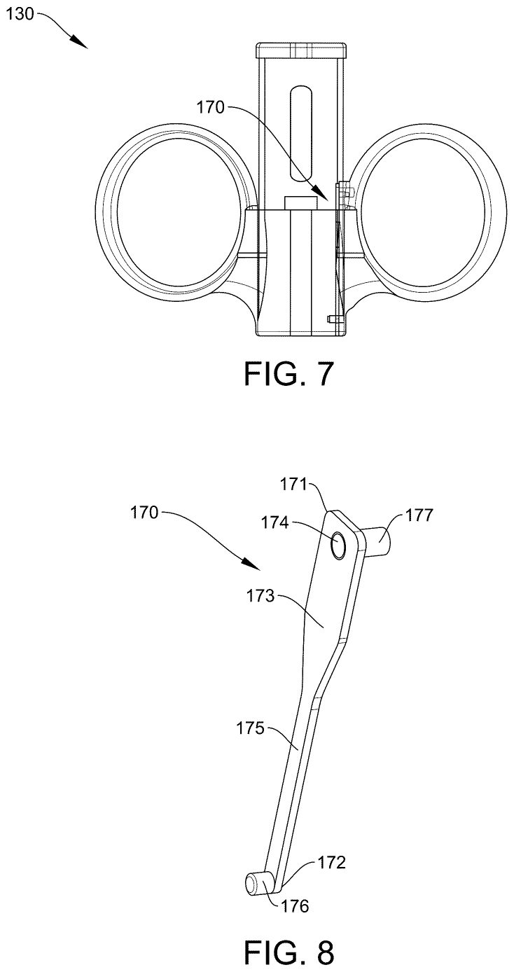

According to various exemplary embodiments, the handle 100 comprises a locking and tracking mechanism for the first control members. This mechanism includes a flexible member 170, otherwise referred to as a flexure with a proximal end 171 coupled to the sliding component 130 and a distal end 172 slidably coupled to an exterior of the handle body 102 via a track 110 formed in the exterior surface of the handle body 102. As shown in FIG. 5, the sliding component 130 includes a part 144 extending off a radially inward side of the second ring 143, the part including a pin hole 145 for coupling to the flexible member 170. As shown in FIGS. 6-7, the flexible member 170 is coupled to the sliding component 130 via a proximal pin 177 projecting laterally outward from an enlarged proximal portion 173 into the pin hole 145 of the part 144.

As shown in FIG. 8, the flexible member 170 of this embodiment has an enlarged proximal portion 173 that tapers to a narrower distal portion 175. In an embodiment, the distal end of the flexible member 170 comprises a distal pin 176 sized and shaped to fit within the track 110. In an embodiment, the enlarged proximal portion 173 includes a pin hole 174 within which the proximal pin 177 is rotatably received so that the enlarged proximal portion 173 is rotatably coupled to the proximal pin 177. As indicated above, the proximal pin 177 is sized and shaped to be received in the pin hole 145 of the second ring 143 of the body portion 133 of the sliding component 130. Accordingly, the flexible member 170 is rotatably coupled to the sliding component 130 via the proximal pin 177. In other embodiments, the proximal pin 177 can be formed with the flexible member 170, and the proximal pin 177 can be permitted to rotate within the pin hole 174 to rotatably couple the flexible member 170 to the sliding component 130. It should be understood that only minor degrees of rotation are permitted to the flexible member 170 relative to the sliding component 130 in its intended configuration (e.g., only a few degrees of rotation) to permit the flexible member 170 to travel within a proximal portion 112 of the track 110 as will be explained in greater detail below.

The flexible member 170 is shaped with the enlarged proximal portion 173 to allow space for the pin hole 174 and to further affect the flexibility of the proximal portion, e.g., encouraging flexing about a transverse axis. In an embodiment, a radially outward force imposed on the distal end 172 of the flexible member 170 bends the flexible member 170. A radially inward face of the flexible member 170 faces the body portion 133 of the sliding component 130 and the exterior surface of the handle body 102. In an intended configuration, the distal pin 176 is received within the track 110. Due to the coupling of the flexible member 170 to the sliding component 130, the motion of the distal pin 176 within the track 110 is directly correlated to the motion of the first control members (e.g., extension members 230) and, by extension, the configuration of the clip (e.g., the clip 210) at the distal end of the device.

The track 110 is formed on a surface on the exterior of the handle body 102. The handle body 102 can be formed so that the surface is flat and of sufficient thickness for the track 110 to be cut thereinto without penetrating into the channel 106 of the handle body 102. The track 110 generally comprises a slot sized and shaped to receive a distal pin 176 of a flexible member 170. As the sliding component 130 and the proximal end 171 of the flexible member 170 move longitudinally the distal pin 176 travels along the track 110 (i.e., travels within the slot). The track 110 is shaped with features having a variable depth, wherein certain locations within the slot are shaped to guide or restrict the motion of the distal pin 176 therewithin. In one example, a longitudinal force can be imparted upon the sliding component 130 to translate the sliding component 130 along the handle body 102, accordingly moving the distal pin 176 of the flexible member 170 along the track 110. Conversely, a feature of the track 110 that resists motion of the distal pin 176 within the slot imparts a force upon the sliding component 130 resisting the translation of the sliding component 130 and, by extension, resisting the translation of the control members attached thereto (e.g., the extension members 230).

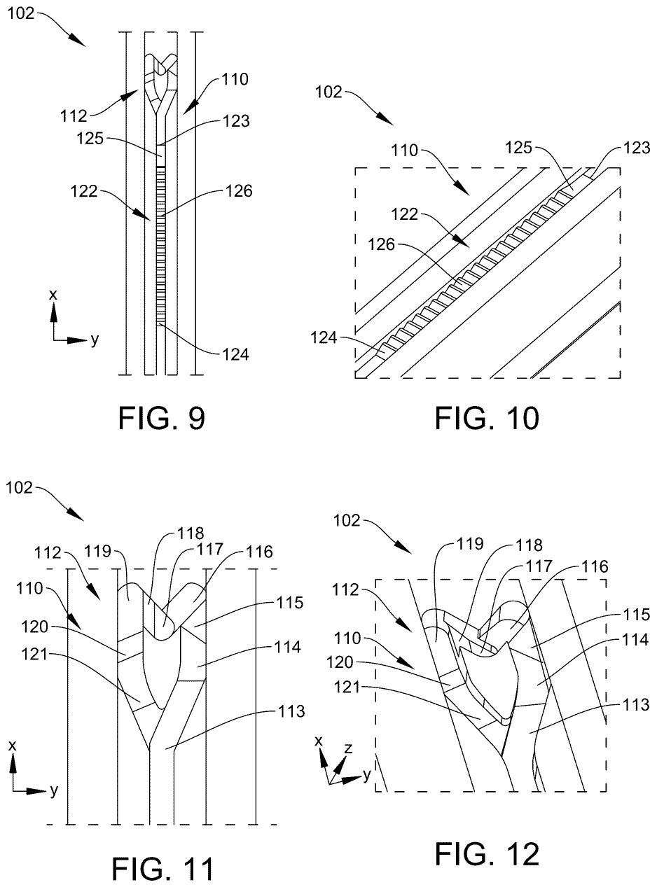

As shown in FIGS. 9-18, the track 110 of this embodiment generally comprises a proximal portion 112 including a loop and a ridged portion 122 including a series of ridges 126. For ease of description, FIGS. 9, 11 include an axis indicator showing an x-axis and y-axis and FIG. 12 includes an axis indicator showing an x-axis, y-axis and z-axis, wherein the x-axis corresponds to the longitudinal axis of the handle body 102, and the y-axis and z-axis correspond to axes transverse to the longitudinal axis. It should be understood that the z-axis generally corresponds to a depth of the track 110, as shown in greater detail in FIG. 12.

The proximal portion 112 is shaped so that the distal pin 176 generally travels in one direction around the loop (e.g., counter-clockwise) and includes a part that, when engaged with the distal pin 176 of the flexible member 170, provides a temporary lock. In other words, when the distal pin 176 is engaged with the locking location, the locking location retains the distal pin 176 therein so that the sliding component 130 and, consequently, the clip 210 are retained in a set configuration (e.g., the insertion configuration) without requiring active user control to maintain the configuration, e.g., to prevent the sliding component 130 from slipping relative to the handle body and transitioning the clip toward a different operating configuration.

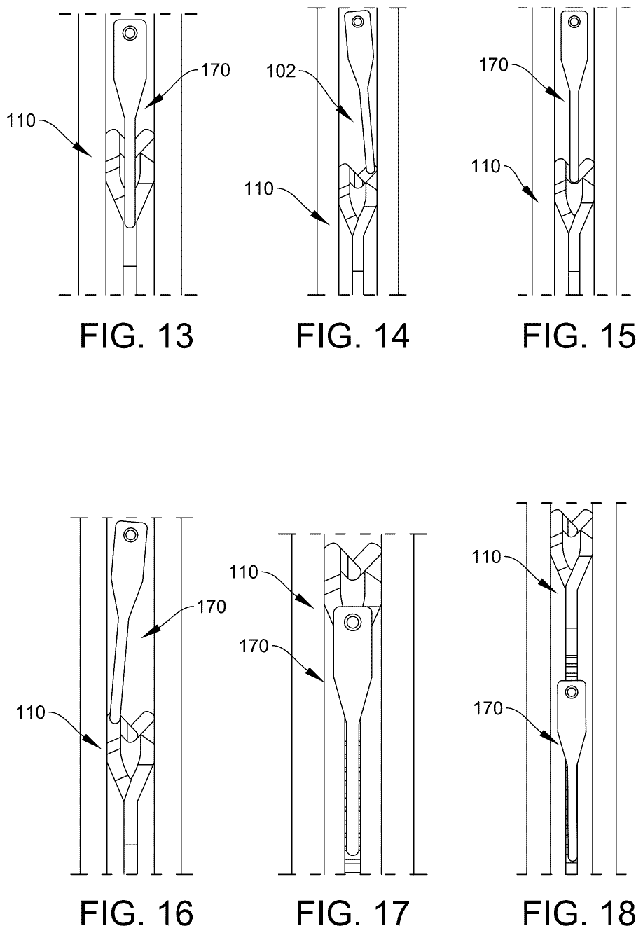

Starting at the bottom of the loop and progressing in a generally counter-clockwise direction, the loop comprises a first part 113, a second part 114, a third part 115, a fourth part 116, a fifth part 117, a sixth part 118, a seventh part 119, an eighth part 120, and a ninth part 121. The parts 113-121 are subdivided as such due to the differing depths of the parts 113-121. The first part 113 comprises a curved part that starts the loop, as shown in FIG. 13. The first part 113 is flat. The second part 114 comprises a longitudinal part that decreases in depth (i.e., elevates radially outward in the z-direction). The third part 115 comprises a longitudinal part that is flat. After the third part 115 is a drop-off into the fourth part 116. Accordingly, as the distal pin 176 progresses past the third part 115, the distal pin 176 suddenly drops into the fourth part 116 and is not permitted to travel back to the third part 115, as shown in FIG. 14. In this embodiment, the distal pin 176 is engaged with the fourth part 116 at a top of a proximal stroke of the sliding component 130.

The fourth part 116 comprises a part angled radially inward and distal (in the xy-plane) and which decreases in depth (i.e., elevates radially outward in the z-direction). After the fourth part 116 is a drop-off into the fifth part 117. Accordingly, as the distal pin 176 progresses past the fourth part 116, the distal pin 176 suddenly drops into the fifth part 117 and is prevented from traveling back to the fourth part 116, as shown in FIG. 15. The fifth part 117 is a flat part. The sixth part 118 is angled radially outward and proximally (in the xy-plane) and decreases in depth (i.e., elevates radially outward in the z-direction). Accordingly, when the distal pin 176 is engaged with the fifth part 117 it is in a locked configuration locking a position of the sliding component 130 as the distal pin 176 cannot travel back to the fourth part 116 and needs to overcome an incline of the sixth part 118. The fifth part 117 can be considered a locking location for the sliding component 130. This position of the sliding component 130 corresponds in this embodiment to the insertion configuration of the clip.

The user can draw the sliding component 130 distally to bring the distal pin 176 past the sixth part 118 into a seventh part 119, as shown in FIG. 16. The seventh part 119 is a flat part. The eighth part 120 is a curved part that decreases in depth (i.e., elevates radially outward in the z-direction). The ninth part 121 is a flat part that completes the loop. After the ninth part 121 is a drop-off into the first part 113, bringing the distal pin 176 back to the position of FIG. 13. Accordingly, to return to, e.g., the locked position, the distal pin 176 must travel around the loop again as it is restricted from entering the loop at the ninth part 121. The above features are possible due to the flexible nature of the flexible member 170, which permits the distal pin 176 to move radially outward or inward to conform to the variable depth of the track 110.

Distal to the proximal portion 112 of the track 110 is the ridged portion 122 of the track 110. As shown in FIGS. 9-10, the ridged portion 122 extends from a proximal end 123 to a distal end 124 and comprises a first inclined portion 125 that decreases in depth and leads to a series of ridges 126. As the distal pin 176 travels distally over the track 110 it encounters the series of ridges 126, as shown in FIG. 17. As it progresses over the series of ridges 126, each ridge 126 provides tactile feedback to the user corresponding to the distance travelled by the distal pin 176. The ridges 126 provide a small resistive force to the distal pin 176 traveling within the track 110, such that as each ridge 126 is overcome, the user is made aware of the distal progression. The proximal end of the series of ridges 126 can correspond to the initial deployment configuration of the clip and the distal end of the series of ridges 126 can correspond to the review configuration of the clip. Accordingly, as the clip is separated from the endoscope, the user is made aware of the distal progression of the clip relative to the endoscope.

As shown in FIG. 1, the handle 100 further includes a deployment feature 150 slidably coupled to the handle body 102 between the proximal ring 140 and the body portion 133 of the sliding component 130. The deployment feature 150 can travel distally and proximally along the handle body 102 to control a position of second control members fixed to the deployment feature 150 and extending distally therefrom. In particular, the second control members of this embodiment correspond to the release members 250 of the clipping system 200 that function to couple the clip 210 to the extension members 230 throughout transitions between the insertion configuration, the initial deployment configuration and the review configuration and further function to decouple the clip 210 from the extension members 230 and a remainder of the clipping system 200 in a final deployment of the clip Accordingly, prior to final deployment of the clip, the deployment feature 150 generally follows the sliding component 130 such that the second control members remain in the same position relative to the first control members and the clip remains coupled to the clipping system. Upon satisfactory placement of a clip, the user can actuate the deployment feature 150 to retract the second control members to finally deploy the clip 210 as will be described in greater detail below.

In an embodiment, the location of the deployment feature 150 along the handle body 102 generally follows the location of the sliding component 130 along the handle body 102, i.e., proximal or distal motion of the sliding component 130 generally causes a proximal or distal force to be imparted on the deployment feature 150 sufficient to move the deployment feature 150 in a manner comparable to the sliding component 130. However, the location of the deployment feature 150 relative to the sliding component 130 is generally not fixed, i.e., the deployment feature 150 and the sliding component 130 are partially decoupled such that the deployment feature 150 can be located closer to the proximal ring 140 or closer to the body portion 133, i.e., the deployment feature 150 can move/slide relative to the sliding component 130.

A spring 182 constrains the motion of the deployment feature 150 relative to the sliding component 130, as described below. Additionally, the deployment feature 150 of this embodiment is coupled to the sliding component 130 via a slot 139 in the back portion 138 of the sliding component 130. A pin extends through the slot 139 to connect to the deployment feature 150. The pin travels within the slot 139, such that the relative positions between the deployment feature 150 and the sliding component 130 is constrained by the length of the slot 139.

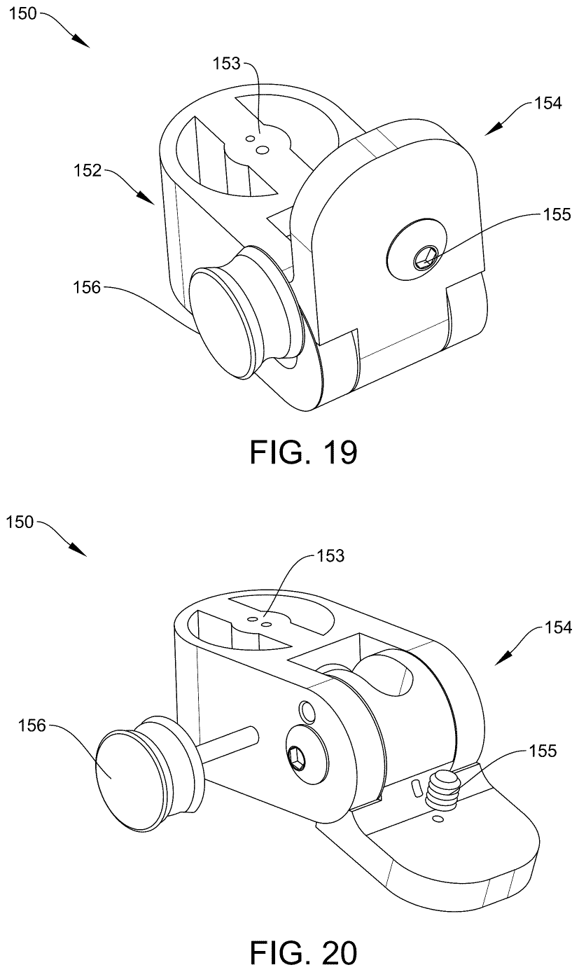

As shown in FIGS. 19-20, the deployment feature 150 generally comprises a body portion 152 having a cross-section similar to that of the body portion 133 of the sliding component 130, such that the deployment feature 150 can slide along the handle body 102. The body portion 152 includes channels 153 through which the second control members extend. The body portion 152 includes a rotatable switch 154 rotatably coupled thereto via pins. The switch 154 includes a screw 155 or a pin coupled to the switch 154 that holds a proximal end of the second control members. The switch 154 can be held in position, i.e., prevented from rotating, by a pin 156. When the user determines to deploy the clip, the pin 156 may be removed so that the switch 154 can be rotated to pull the second control members proximally, as shown in FIG. 20.

As shown in FIGS. 21-22, the deployment feature 150 of this embodiment is coupled to the sliding component 130 by a spring 182, e.g., a compensation spring. The spring 182 partially decouples the motion of the sliding component 130 and the deployment feature 150, such that the deployment feature 150 can move away from the sliding component 130, as shown in FIG. 21, or move close to the sliding component 130, as shown in FIG. 22. The spring 182 compensates for differing lengths of the first and second control members as the endoscope navigates tortuous body lumens and while transitioning the clip between its operating configurations. It is essential that the second control members for deploying the clip are not withdrawn relative to the clip prior to final deployment. Accordingly, if the second control members need some additional length to remain in position during the operation of the clip, the deployment feature 150 is drawn closer to the sliding component 130 as the spring 182 is compressed.

It will be apparent to those skilled in the art that various modifications may be made in the present disclosure, without departing from the scope of the disclosure. Furthermore, those skilled in the art will understand that the features of any of the various embodiments may be combined in any manner that is not inconsistent with the description and/or the functionality of the embodiments.

1-15. (canceled)

16. A clipping system for treating tissue, comprising:
a clip mounted over a distal end of an endoscope in an insertion configuration in which jaws of the clip are in an open configuration;
an extension member coupled at a distal end thereof to the clip, the extension member movable relative to the endoscope to move the clip off the distal end of the endoscope to transition the clip into an initial deployment configuration in which the jaws of the clip are in a closed configuration and to separate the clip in the closed configuration from the distal end of the endoscope by a distance to transition the clip into a review configuration; and
a handle comprising:
a handle body including an exterior surface shaped for gripping by an operating physician, the handle body including a longitudinal slot extending therethrough;
a sliding component slidably coupled to the handle body by a body portion including a transverse portion extending through the longitudinal slot of the handle body, a proximal end of the extension member coupled to the sliding component so that longitudinal motion of the sliding component along the handle body moves the extension member to transition the clip between the insertion configuration, the initial deployment configuration and the review configuration; and
a flexible member coupled at a proximal end to the sliding component and movable therewith, the flexible member extending along the exterior surface of the handle body to a distal end comprising a distal pin extending toward the exterior surface of the handle body,
wherein the exterior surface of the handle body further includes a track sized and shaped for the distal pin of the flexible member to travel therewithin, the track comprising a depth that is non-uniform, the track including variable depth features at locations along the track for resisting movement of the distal pin within the track so that, as the sliding component moves along the handle body and the distal pin of the flexible member travels within the track, the distal pin encountering the variable depth features along the track provides feedback regarding a position of the clip relative to the endoscope.

17. The clipping system of claim 16, wherein the flexible member is unflexed or at a minimum degree of flexing when the distal pin is engaged with the track in a location of maximum depth, wherein the distal pin moving to locations of lesser depth within the track causes the flexible member to flex radially outward relative to the handle body.

18. The clipping system of claim 16, wherein the track includes a locking location wherein, when the distal pin is within the locking location, the distal pin is impeded from traveling outside the locking location such that, absent a force being applied to the sliding component by a user, the distal pin remains in the locking location and the clip remains in position relative to the endoscope.

19. The clipping system of claim 18, wherein the track includes a proximal portion shaped in a loop, wherein the proximal end of the flexible member is rotatably coupled to the sliding component to permit the flexible member to rotate out of longitudinal alignment with the handle body and permit the distal pin to travel within the loop.

20. The clipping system of claim 19, wherein the depth of the proximal portion of the track is configured to urge the distal pin to travel in only one direction around the loop.

21. The clipping system of claim 20, wherein first parts of the loop are angled to reduce the depth of the track and second parts of the loop include drop-offs to increase the depth of the track such that, after the distal pin travels in a first direction around the loop past a drop-off, the distal pin is restricted from traveling in a second direction around the loop opposite the first direction.

22. The clipping system of claim 19, wherein the locking location is within the proximal portion of the track and corresponds to the insertion configuration of the clip, the locking location being distal to parts of the loop on either side the locking location so that the distal pin can exit the locking location by the user withdrawing the sliding component further proximally, causing the distal pin to travel proximally to a drop-off, whereupon the distal pin can travel distally out of the loop.

23. The clipping system of claim 16, wherein the track includes a ridged portion comprising a series of ridges so that, as the distal pin travels along the ridged portion, tactile feedback is imparted to the handle.

24. The clipping system of claim 23, wherein the distal pin traveling distally over the ridged portion corresponds to transitioning the clip from the initial deployment configuration to the review configuration.

25. The clipping system of claim 16, further comprising:
a release member in a retaining position configured to retain a coupling of the clip with the clipping system, the release member movable to a release position in which the release member is withdrawn proximally to remove the coupling and the clip to decouple from the clipping system.

26. The clipping system of claim 25, further comprising:
a deployment feature to which a proximal end of the release member is fixed, the deployment feature including a switch operable to withdraw the release member proximally relative to the clip.

27. The clipping system of claim 26, further comprising:
a spring comprising a proximal end coupled to the deployment feature and a distal end coupled to the sliding component so that motion of the sliding component is partially decoupled from motion of the deployment feature.

28. The clipping system of claim 27, wherein the sliding component includes a back portion extending proximally from the body portion and a proximal ring at a proximal end, the deployment feature positioned between the proximal ring and the body portion of the sliding component restricting the relative motion between the deployment feature and the sliding component.

29. The clipping system of claim 28, wherein the back portion of the sliding component includes a slot, a pin extending through the slot and connecting to the deployment feature to constrain a relative distance between the deployment feature and the body portion of the sliding component.

30. The clipping system of claim 28, wherein the relative distance between the deployment feature and the body portion of the sliding component can vary during operation of the clip.

31. A method for treating tissue, comprising:
inserting, to a target area in a body lumen, a clip mounted over a distal end of an endoscope in an insertion configuration in which first and second jaws of the clip are in an open configuration, an extension member coupled at a distal end thereof to the clip, the extension member movable relative to the endoscope by a handle comprising a handle body including an exterior surface shaped for gripping by an operating physician, the handle body including a longitudinal slot extending therethrough, the handle comprising a sliding component slidably coupled to the handle body by a body portion including a transverse portion extending through the longitudinal slot of the handle body, a proximal end of the extension member coupled to the sliding component so that longitudinal motion of the sliding component along the handle body moves the extension member, the handle comprising a flexible member coupled at a proximal end to the sliding component and movable therewith, the flexible member extending along the exterior surface of the handle body to a distal end comprising a distal pin extending toward the exterior surface of the handle body, wherein the exterior surface of the handle body further includes a track sized and shaped for the distal pin of the flexible member to travel therewithin, the track comprising a depth that is non-uniform, the track including variable depth features at locations along the track for resisting movement of the distal pin within the track so that, as the sliding component moves along the handle body and the distal pin of the flexible member travels within the track, the distal pin encountering the variable depth features along the track provides feedback regarding a position of the clip relative to the endoscope;
drawing tissue between the first and second jaws of the clip;
sliding the sliding component distally to move the extension members distally to move the clip distally off of the distal end of the endoscope to transition the clip into an initial deployment configuration in which the first and second jaws of the clip are in a closed configuration around the tissue; and
sliding the sliding component further distally to move the extension members further distally to separate the clip in the closed configuration from the distal end of the endoscope by a distance to transition the clip into a review configuration,
wherein the distal pin encounters the variable depth features along the track during the sliding of the sliding component to provide feedback regarding transitions between the insertion configuration, the initial deployment configuration and the review configuration.

32. The method of claim 31, wherein the flexible member is unflexed or at a minimum degree of flexing when the distal pin is engaged with the track in a location of maximum depth, wherein the distal pin moving to locations of lesser depth within the track causes the flexible member to flex radially outward relative to the handle body.

33. The method of claim 31, wherein the track includes a locking location wherein, when the distal pin is within the locking location, the distal pin is impeded from traveling outside the locking location such that, absent a force being applied to the sliding component by a user, the distal pin remains in the locking location and the clip remains in position relative to the endoscope.

34. The method of claim 33, wherein the track includes a proximal portion shaped in a loop, wherein the proximal end of the flexible member is rotatably coupled to the sliding component to permit the flexible member to rotate out of longitudinal alignment with the handle body and permit the distal pin to travel within the loop.

35. The method of claim 31, wherein the track includes a ridged portion comprising a series of ridges so that, as the distal pin travels along the ridged portion, tactile feedback is imparted to the handle.
